(19) United States
(12) Patent Application Publication
(10) Pub. No.: US 2010/0056936 A1
(43) Pub. Date: Mar. 4, 2010

(54) BLOOD FLOW RATE IMAGING DEVICE

(76) Inventors: Hitoshi Fujii, Kitakyushu-shi (JP); Kenji Okamoto, Iizuka-shi (JP); Kimihiko Fujisawa, Fukuoka-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 12/517,017

(22) PCT Filed: Nov. 28, 2007
(86) PCT No.: PCT/JP2007/072907

§ 371 Date: Nov. 2, 2009

(30) Foreign Application Priority Data
Dec. 1, 2006 (JP) 2006-326370

(51) Int. Cl. A61B 5/0265

(52) U.S. Cl. 600/504

(57) ABSTRACT

Provided is a blood flow rate imaging device that can automatically distinguish an artery and a vein from a blood flow rate obtained on a time series blood flow map. A blood flow rate imaging device of the present invention includes a laser beam irradiation system (1) that irradiates a laser beam to a biological tissue that has a blood cell, a light receiving system (2) that has a light receiver (5) including many pixels that detect reflected light from the biological tissue, an image capture section (12) that continuously captures a plurality of images for a predetermined time of one or more cardiac beats on the basis of signal from the light receiver (5), an image storage section (15) that stores a plurality of images, and an arithmetic section (16) that calculates a blood flow rate within a biological tissue from the time variation of the output signal of each pixel corresponding to a plurality of stored images, wherein the above arithmetic section has a detecting section that detects an artery and a vein from a plurality of images of the above one or more cardiac beats and displays an arterial pulse part and a venous pulse part on the blood flow map.

### TECHNICAL FIELD

The present invention relates to a blood flow rate imaging device for measuring and imaging a blood flow rate on the basis of a speckle signal reflected from a biological tissue obtained by irradiating the biological tissue having blood cells with a laser beam.

### BACKGROUND ART

A blood flow rate measuring instrument is conventionally known that irradiates with a laser beam a biological tissue having blood cells such as an eye ground of a subject's eye, introducing images formed by reflected light from the blood cells onto an image sensor such as a solid-state image sensor (CCD or CMOS), sequentially captures and stores a large number of these images at predetermined time intervals, selects a predetermined number of images from the large number of the stored images, calculates a value obtained by accumulating amounts of time variation of output in each pixel of each image and then calculates the velocity of blood cells (blood flow rate) from the value. In addition, in this kind of blood flow rate measuring instrument, the amount of the output variation of each pixel corresponds to the movement speed of the blood cell. Accordingly, a blood flow distribution in a biological tissue can also be color-displayed on a monitor screen as a two-dimensional image (blood flow map) on the basis of the value of the output variation of each of these calculated pixels, and this device is, for example, put to practical use as an inspection device of the blood flow of the eye ground.
[Patent Document 1]: Japanese Examined Patent Application Publication No. Hei 5-28133
[Patent Document 2]: Japanese Examined Patent Application Publication No. Hei 5-28134
[Patent Document 3]: Japanese Unexamined Patent Application Publication No. Hei 4-242628
[Patent Document 4]: Japanese Unexamined Patent Application Publication No. Hei 8-112262
[Patent Document 5]: Japanese Unexamined Patent Application Publication No. 2003-164431
[Patent Document 6]: Japanese Unexamined Patent Application Publication No. 2003-180641

In conventional blood flow rate measuring instruments, however, a blood flow map has been only observed by means of a moving image and a physical quantity that characterizes a change in blood flow has not been examined yet. Even in conventional ones also, although the blood flows of blood streams and tissue blood flows can be ascertained on a map, whether or not they are attributed to arterial pulses or venous pulses has been uncertain. The time dependency of a blood flow must be analyzed in order to distinguish whether the pulse is due to an arterial pulse or a venous pulse. However, such analysis has been difficult by use of conventional blood flow rate measuring instruments. In other words, the pulse of the blood flow of each pixel of each image measured by conventional blood flow rate measuring instrument is a blood flow data including a statistical error that scatters around a certain blood flow value. On account of this, the data does not become a clear pulse profile, but a profile with many noises, when the pulses are arranged in a time series. Thus, it has been extremely difficult to detect the peak time of a pulse needed for dividing arterial and venous pulse regions.

### DISCLOSURE OF THE INVENTION

An object of the present invention is to provide a blood flow rate imaging device that can apply and develop a conventional blood flow rate measuring instrument, suppress noises of a blood flow pulse data with many noises and display arterial pulse and venous pulse parts on a map.

The present inventor has successfully developed a method and an apparatus that analyzes a change in blood flow that appears at regular intervals synchronously with cardiac beats in each site within an observation field of view for a series of blood flow maps obtained in blood flow measurement for a few seconds, introduces a numerical value that can distinguish between an arterial site with a sharp rise waveform and a venous site with a waveform gradually going up and down, and distinguishes both the sites and displays a two-dimensional map to thereby determine which site possibly dangerously becomes an ischemic state.

An invention described in claim 1 of the present invention is a blood flow rate imaging device; comprising: a laser beam irradiation system that irradiates a biological tissue having a blood cell with a laser beam; a light receiving system having a light receiver including a large number of pixels that detects reflected light from the above biological tissue; an image capture section that continuously captures a plurality of images for a specified time that is one or more cardiac beats on the basis of a signal from the above light receiver; an image storage section that stores the above plurality of images; an arithmetic section that calculates a blood flow rate within the biological tissue from the time variation of the output signal of each pixel corresponding to the plurality of the stored images; and a display section that displays the two-dimensional distribution of the calculation result as a blood flow map, wherein the above arithmetic section has a detecting section that detects an artery and a vein from a plurality of images of the above one or more cardiac beats and distinguishably displays an arterial pulse part (artery map) and a venous pulse part (vein map) on the blood flow map of the above display section.

In the present invention, so long as an artery map and a vein map are distinguishably displayed on a blood flow map, the way of displaying an artery and vein map on a blood flow map is by no means limited. A blood flow map can be, for example, superimposed upon (invention described in claim 9), arranged with, sidably superimposed upon, or combined with, an artery and vein map to be thereby displayed. In addition, it is needless to say that a well-known mechanism or a means can be added to or incorporated into a blood flow rate imaging device of the present invention as required.

The invention described in claim 2 is the blood flow rate imaging device described in claim 1, wherein the above detecting section calculates skewness (skew value) based on the variation of blood flow rates arranged in a time series for each pixel and detects an arterial pulse part and a venous pulse part.

The invention described in claim 3 is the blood flow rate imaging device described in claim 1, wherein the above detecting section calculates the expected value of a probability density function by likening the variation of blood flow rates arranged in a time series for each pixel to the probability density function and detects an arterial pulse part and a venous pulse part.

The invention described in claim 4 is the blood flow rate imaging device described in claim 1, wherein the above detecting section calculates kurtosis based on the variation of blood flow rates arranged in a time series for each pixel and detects an arterial pulse part and a venous pulse part.

The invention described in claim 5 is the blood flow rate imaging device described in claim 1, wherein the above detecting section calculates a mode in which a probability density function is estimated to be a maximum by likening the variation of blood flow rates arranged in a time series for each pixel to a probability density function and detects an arterial pulse part and a venous pulse part.

The invention described in claim 6 is the blood flow rate imaging device described in any one of claims 2 to 5, wherein the above detecting section statistically processes a peripheral blood flow value of one or more pixels for the blood flow value of each pixel including many statistical errors to calculate an average value and outputs one or more pulse components arranged in a time series with few noises needed for detecting the arterial pulse part and the venous pulse part.

The invention described in claim 7 is the blood flow rate imaging device described in any one of claims 2 to 5, wherein the above detecting section averages time variations of the blood flow of each pixel over a plurality of cardiac beats for one cardiac beat and then extracts the pulse component.

The invention described in claim 8 is the blood flow rate imaging device described in any one of claims 2 to 5, wherein the above detecting section cuts out one cardiac beat for the time variations of the blood flow of each pixel over a plurality of cardiac beats, for example, based on a synchronization signal from the outside that synchronizes with a cardiac beat such as from an electrocardiograph and then extracts a pulse component.

Additionally, the invention described in claim 9 is the blood flow rate imaging device described in claim 1, wherein, in the above display, an arterial pulse part is superimposed upon a venous pulse part on the blood flow map and displayed. The invention described in claim 9 displays a blood flow map to be superimposed upon an artery and vein map in the above display and this invention case includes distinguishably displaying an arterial pulse part and a venous pulse part on the blood flow map. Moreover, it is needless to say that the technical feature of displaying the superimposition of an arterial pulse part upon a venous pulse part on the blood flow map can be combined with other inventions described in any of claims 2 to 8 of the present invention.

When the superimposition of the aforementioned blood flow map and artery and vein map is displayed, the ratio may not be 1:1, and the amount of each map may be multiplied by a certain numerical value, i.e., a weight in order to more clearly observe ischemic states. As a result of the weight multiplication, for example, the addition of a slow part of a blood flow map to the vein site of an artery and vein map makes it possible to recognize the site of an ischemic state on the eye ground.

In the blood flow rate imaging device of the present invention, the detecting section has been specifically made up like the invention described in claims 2 to 8 and therefore an arterial pulse part can be distinguished from a venous pulse part to obtain the effect of acquiring a classified, easily understandable classification map, from a plurality of blood flow maps of one or more cardiac beats.

In a blood flow map obtained by a device of the present invention, a site that indicates a venous pulse and has a low blood flow shows a disorder and the display visualization of this site is medically significant.

In addition, the adoption of the way of display like the invention described in claim 9 allows the following effects to be obtained. For example, the venous pulse can be shown in black and the arterial pulse in red, while a fast blood flow site becomes white and a slow blood flow site becomes black when the blood flow map is indicated by a grey scale map. Thus, when the superimposition of a blood flow map upon an artery and vein map is employ for display, a site that is low in blood flow and shows a disorder becomes black on the blood flow map and becomes a venous pulse and black also in an artery and vein map. Consequently, display by making an artery and vein map penetrated to some extent and superimposing makes a disorder site shown in black easily understandable.

In the above way of superimposition for display, when maps are superimposed upon each other, the feature is that a colored artery and vein map is translucently penetrated and a blood flow map is superimposed thereupon in a gray scale (black and white) for display. In this case, a site that is black in the artery and vein map is a place where a venous pulse, particularly a pulse peak, is slow and a site in which some trouble is doubtfully caused. However, a colored site that is not black has a shape in which a peak is present before a pulse and can be called a healthy site. On the other hand, the black part of a blood flow map has a considerably slow flow, in which some obstacles are considered to inhibit the blood flow and in which a disorder is also doubtfully caused. In addition, as displayed in a grey scale (black and white), this part is displayed considerably blackish. As such, when a map in which the translucent artery and vein map that is made a colored map is superimposed upon the blood flow map in a grey scale is observed, a darkly displayed part is displayed further dark because each map is dark, whereby a site where some disorder is considered to be caused is clearly displayed.

### BRIEF DESCRIPTION OF THE DRAWINGS

FIG. 1 is a diagram indicating a main part of the constitution of the blood flow rate imaging device of the present invention.

FIG. 2 is a diagram indicating the pulse of the blood flow of each pixel.

FIG. 3 is a diagram indicating a smoothed, normalized pulse.

FIG. 4 is a diagram in which skewness obtained from the present invention was made a map (actually a colored map).

FIG. 5 is a diagram indicating a flow for calculating the skewness.

FIG. 6 is a diagram indicating a flow for calculating simplified skewness.

FIG. 7 is a diagram in which skewness obtained from the present invention was made a black and white map.

FIG. 8 is a diagram indicating a main part of the constitution of a blood flow rate imaging device described in claim 8.

### BEST MODE FOR CARRYING OUT THE INVENTION

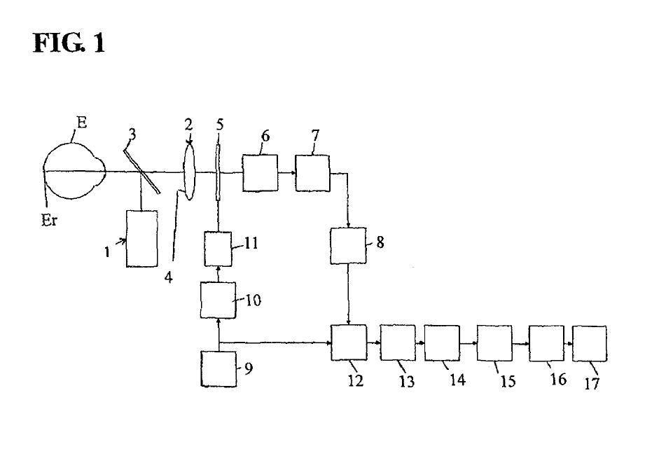

Hereinafter, the present invention will be described with reference to the drawings. FIG. 1 shows an overview of an optical system of the constitutions of a blood flow rate imaging device of the present invention; reference numeral 1 is a laser beam irradiation system, reference numeral 2 is a light receiving system, and E is a subject's eye. The laser beam of the laser beam irradiation system 1 is irradiated, for example, to an eye ground Er as a biological tissue of a subject's eye E, for example, via a half mirror 3.

The light receiving system 2 has a light receiving lens 4, CCD (solid state image sensor) 5 as a light receiver, and an amplifier circuit 6. Laser reflected light from eye ground Er is imaged to CCD 5 as a biological tissue image by the light receiving lens 4. CCD 5 has many pixels on its light receiving face, converts a biological tissue image imaged by the light receiving lens 4 into an electric signal, reads a signal charge by a frame storage system and outputs it as an image signal. The image signal is amplified by a signal amplifier 6, and the image signal amplified by the signal amplifier 6 is output to an analog processing means 7 that carries out, for example, gain control and is converted into a digital signal by an AID converter 8.

Reference numeral 9 is a timing pulse generator, reference numeral 10 is an electronic shutter control means, and reference numeral 11 is a solid state image sensor drive means; the timing pulse generator 9 outputs a timing pulse to the electronic shutter control means 10 and the signal selection tool 12. The solid state image sensor drive means 11 is driven on the basis of the timing pulse.

The signal selection tool 12 receives a digital signal as an image signal A/D converted by the A/D converter 8 and the signal selection tool 12 is recorded in an image recording means 13 on the basis of a timing pulse from the timing pulse generator 9. The image recording means 13 functions as an image capture section that captures a plurality of images at specified time intervals.

Images captured to the image recording means 13 is combined by a blood flow map combining means 14 and is made, for example, one frame image photographed at 1/30 second intervals. The one frame image data is stored in an image storage unit 15 as an image storage section.

Amage signal stored in this image storage unit 15 is input into an arithmetic section 16, and the arithmetic section 16 executes arithmetic processing described below. In addition, reference numeral 17 is a TV monitor as a display portion.

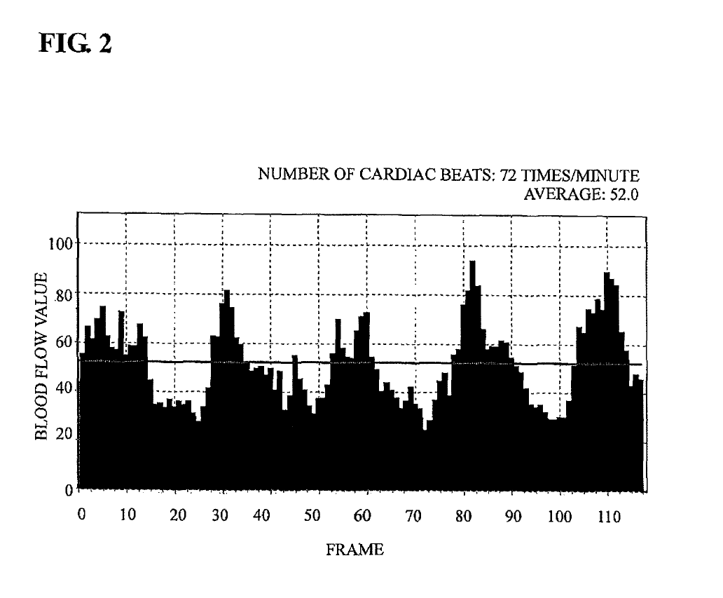

FIG. 2 shows a waveform of the pulse data of each pixel obtained by the blood flow rate imaging device of the present invention. The abscissa represents time, and the ordinate represents blood flow values.

In order to divide arterial pulse and venous pulse parts from a blood flow map, a method is devised that traces the time variation of blood flow for each pixel in a plurality of continuous blood flow maps of one or more cardiac beats, detects a part that becomes a maximum peak, and regards a part in which its maximum peak time is early as an arterial pulse part and a late part as a venous pulse part. A blood flow obtained by a blood flow meter has a large dispersion due to statistical errors, so that it is extremely difficult to detect the pulse peak for each pixel.

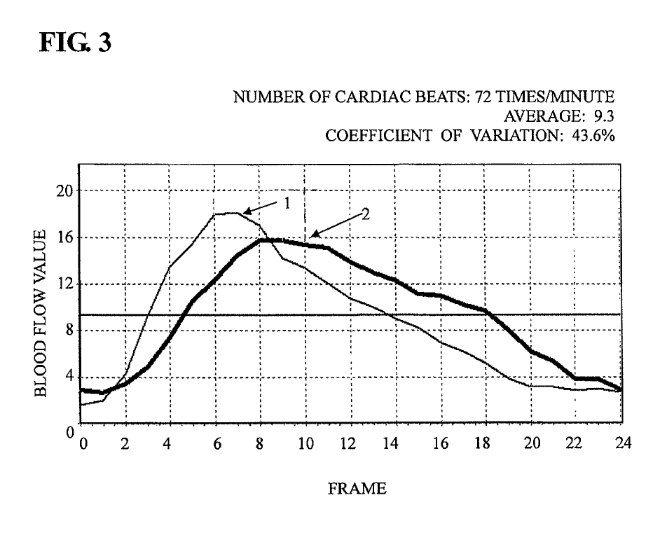

Hence, the present invention focused on being capable of dividing arteries and veins from a profile of a rise till a peak and a fall even in a state in which statistical errors are included to some extent for a method of dividing artery and vein parts even in a data with a large statistical error. For that, first, the data with a large dispersion are averaged in a vicinity of each pixel to converge the data, only one cardiac beat was extracted and arranged in a time series, the average value in a certain region was divided into sites corresponding to the artery and the vein, and then the values were plotted on the same graph to obtain a graph as shown in FIG. 3.

There is also a means for averaging only using spatial blood flow values around each pixel (invention described in claim 6) as a means for obtaining a like graph instead of using averaging in the direction of a time series. That is, for a blood flow value of each pixel including many statistical errors, peripheral blood flow values of one or more pixels may be statistically processed to calculate the average and then to output one or more pulse components with few noises necessary to detect an arterial pulse part and a venous pulse part arranged in a time series. In this case, averaging preferably employs the number of pixels as large as possible. However, averaging by use of a large number of pixels also creates the problem of collapsing streams of small blood vessels. Therefore, the number of pixels so as to obtain a waveform as in FIG. 3 as well as to maintain blood streams to some extent is preferably calculated from a pixel number of 36 of a 6 pixels square around a pixel, or the like, when the width of the blood vessel is set to be 12 pixels for example. The shape of a region to be averaged is not a square, but may also be a circle, cruciform or diamond.

When the number of pixels for averaging in order to sufficiently decrease noises of a pulse component is made large, small blood vessels are removed and their blood streams cannot be recognized. This may, however, be sufficient to separate artery and vein properties of a pulse component in a wide region such as a tissue blood flow. With such a tissue blood flow, the number of pixels to be averaged may be large since the structure of the tissue is large. For example, if the structure of a tissue blood flow has a 20 pixels square, the pixel number is calculated from a number of 100 pixels of a 10 pixels square around a certain pixel, or the like. The shape of a region to be averaged is not a square, but may also be a circle, cruciform or diamond.

As is apparent from FIG. 3, the feature is that the rise for an artery (1 of FIG. 3) is steep and the fall is rapid after the peak, while the feature for the vein (2 of FIG. 3) has a slow rise compared with that for the artery and has a rather slow fall also after the peak. Though the fore-and-aft positions of the peaks for the artery and the vein are also different, it is shown that the ways of the rise till the peak and the fall are more different.

The present invention evaluates a difference in the way of the rise of the both, specifically, first, by skewness (skew value) generally called the third-order moment in statistics as one method. Skewness is a parameter that compares the symmetry properties of functions. When applied to a blood flow, this skewness tends to become a large positive value for an arterial pulse and a small value for a venous pulse.

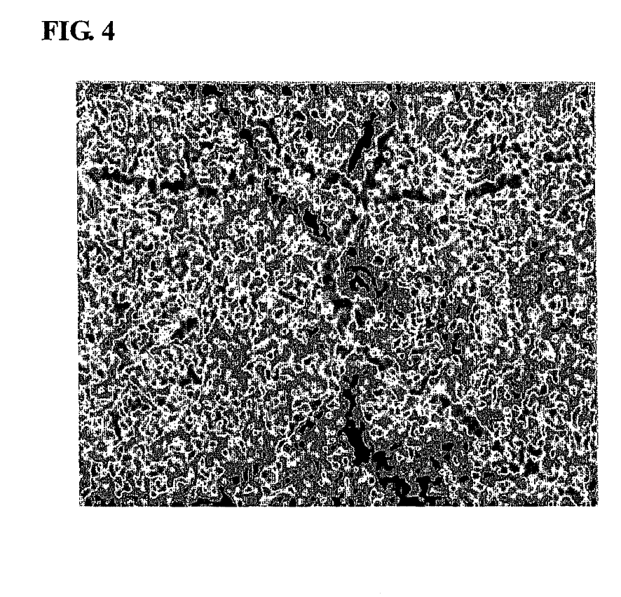

The results in which the skewness was actually calculated and map-displayed were shown in FIG. 4. In FIG. 4, the gray part is an arterial pulse part, and the black part is a venous pulse site. Actually, the part can be color-displayed and as the site becomes red-colored and warmer-colored (gray in FIG. 4), it likely becomes an arterial pulse part, and as the site becomes cold color such as black or blue (black in FIG. 4), it likely becomes a venous pulse site. In addition, the connection of the sites of warm colors ranges like a blood vessel, and the part is thought to be an artery. Moreover, similarly, the connection of sites of cold colors is thought to be a vein.

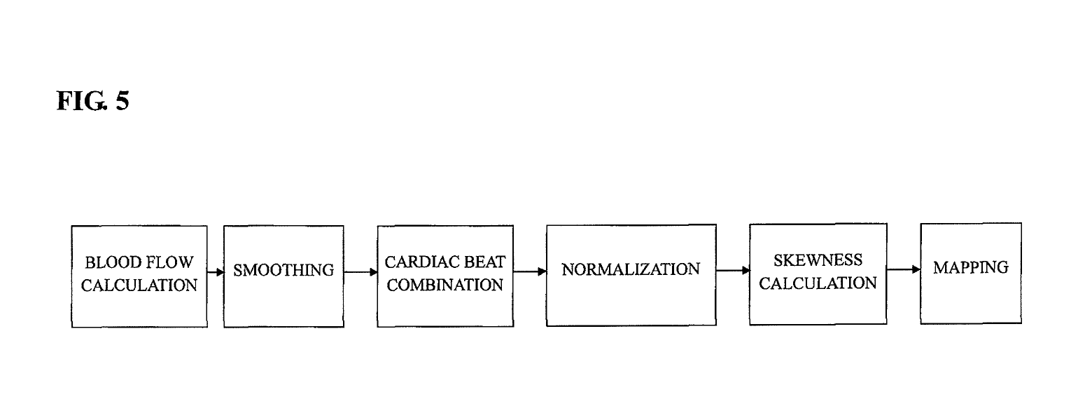

The skewness is actually calculated according to the procedure shown in FIG. 5. In other words, a blood flow value is first calculated from a plurality of speckle images by the blood flow calculation of FIG. 5 to obtain a blood flow map of one or more cardiac beats arranged in a time series. Next, in smoothing, the blood flow values are averaged using peripheral pixels of each pixel for one blood flow map obtained in the above. Then, in cardiac beat combination, the blood flow map of one or more cardiac beats in a time series obtained in the above is detected such that the average blood flow value of the entire map is minimum, to thereby sense a plurality of pulses. The first maps of respective pulses are averaged to make the first map of one cardiac beat subjected to cardiac beat combination. A first map and the subsequent map are sequentially averaged to construct one cardiac beat data subjected to cardiac beat combination.

Next, normalization is performed in the following. Until the above-described procedure, cardiac beat data in each pixel of one cardiac beat are completed; however, the profile of the cardiac beat varies in height with different blood flow values. Thus, the maximum and minimum values of each pixel are sensed and normalization is carried out using the following equation 1 so that pulse profiles can be compared in each pixel. This comes to emphasize the profile of pulses and further emphasize the value of skewness.

$$Ik\_n ( m , n ) = \frac{Ik ( m , n ) - I ( m , n ) min}{I ( m , n ) max - I ( m , n ) min} . \qquad [ Equation 1 ]$$

In the above equation 1, Ik-n(m, n): normalized blood flow value in the k-th map pixel(m, n) from the first cardiac beat map combined to one cardiac beat;
Ik(m, n): blood flow value in the k-th map pixel(m, n) from the first cardiac beat map combined to one cardiac beat;
I(m, n)min: minimum blood flow value, within a time series, of a cardiac beat map combined to one cardiac beat in pixel(m, n); and
I(m, n)max: maximum blood flow value, within a time series, of a cardiac beat map combined to one cardiac beat in pixel(m, n).

Next, for example, skewness is calculated for each normalized pixel of one cardiac beat by applying the following equation 2.

$$Skew ( m , n ) = A {\sum }_{k = 1}^{b} ( {( \frac{k - ave ( m , n )}{stdev ( m , n )} )}^{3} \cdot \frac{Ik\_n ( m , n )}{{\sum }_{l = 1}^{b} Il\_n ( m , n )} ) \qquad [ Equation 2 ]$$

In the above equation 2, Skew (m, n): skewness in pixel (m, n);
A: scale factor; b: number of maps of one cardiac beat; k: k-th map from the first one cardiac beat; ave(m, n): first order moment of a profile in which normalized blood flow values of one cardiac beat are arranged in a time series, generally called the expected value;
stdev(m, n): square root of the second order moment of a profile in which normalized blood flow value of one cardiac beat are arranged in a time series, generally called the standard deviation;
Ik-n(m, n): normalized blood flow value in the k-th map pixel (m,n) from the first cardiac beat map combined to one cardiac beat; and
II-n(m,n): normalized blood flow value in the first map pixel (m,n) from the first cardiac beat map combined to one cardiac beat.

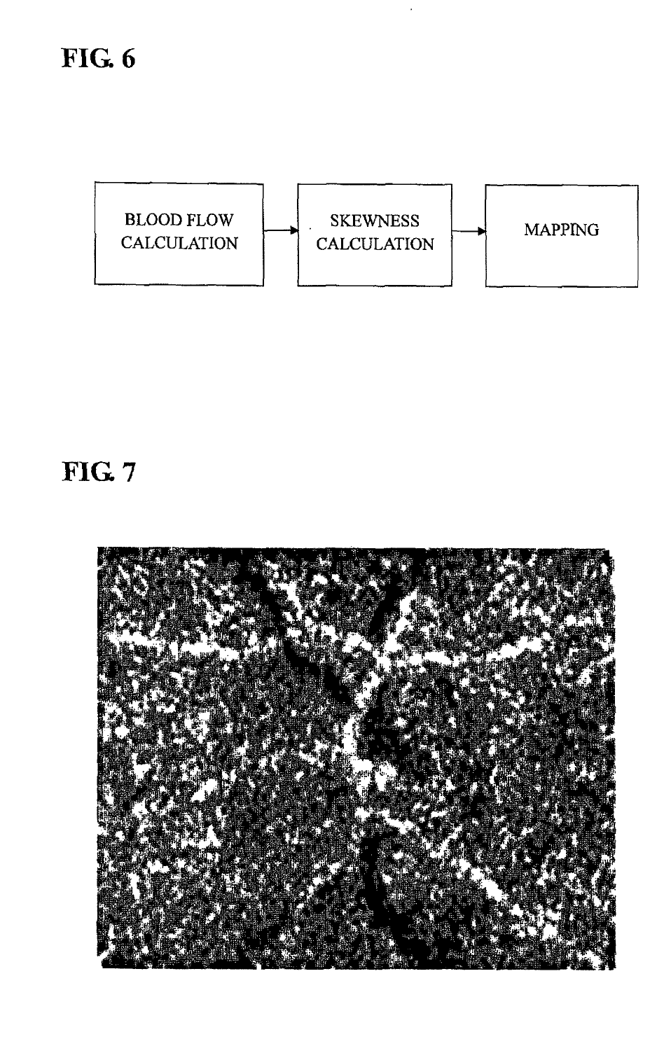

In the present invention, even a method enables artery and vein pulse separation that, in the procedure depicted in FIG. 6 as well, omits on-the-way processes of blood flow and skewness calculations and extracts continuous time series data of only one cardiac beat from a plurality of cardiac beat data to thereby calculate the skewness.

The coefficient A is multiplied such that a user can easily identifies the artery and vein, based on the skewness calculated as described above, thereby making mapping to display a map that divides an artery and vein on a TV monitor or the like. FIG. 7 is a diagram in which the skewness as obtained above was made a black and white map.

A method by skewness described above is an optimum approach as a method of separating arterial and venous pulses. However, the two can be effectively separated also by means of a method using the expected value of the invention described in claim 3 of the present invention, using the skewness of the invention described in claim 4, or using the mode of the invention described in claim 5. The expected value is known as the first moment in statistics, and kurtosis is known as a fourth-order moment.

The expected value is a value inverted by the fore-and-aft position of a pulse peak. The kurtosis has a feature in which the sharper the mode of a pulse, the higher the value and in which the value becomes small if the mode is not sharp. For an arterial pulse, the pulse peak is sharp, so that the value is large. For a venous pulse, the value lowers, so the separation becomes easy.

For the mode, calculation of the mode of the pulse of each pixel is not simply the case because, when the blood flow value of a certain pixel is observed, the pulse profile is not always a clear pulse data because of blood flow value with statistical errors being plotted. Hence, it is preferred that peripheral blood flow values of a pixel is averaged to decrease noises and then the mode is calculated, as in the invention described in claim 6 for calculating a plausible mode. A mode obtained from a pulse profile acquired by averaging each pixel appears in a first portion of the cardiac beat for an arterial pulse and is obtained slightly late for a venous pulse, whereby the arterial pulse can be separated from the venous pulse.

When the number of pixels for averaging is made large in order to maintain the blood stream, a pulse component with a large dispersion including statistical errors is calculated because noises are not sufficiently reduced, so that it may be difficult to execute calculation only by averaging the mode that is a maximum value within a pulse. For example, when a variable component with a faster cycle is present in a pulse component with a slow change, an envelop made up only of peaks with a fast cycle in a pulse component is calculated to estimate an optimal mode and the value of x in which the envelop H(x) is maximum may be set to be the mode.

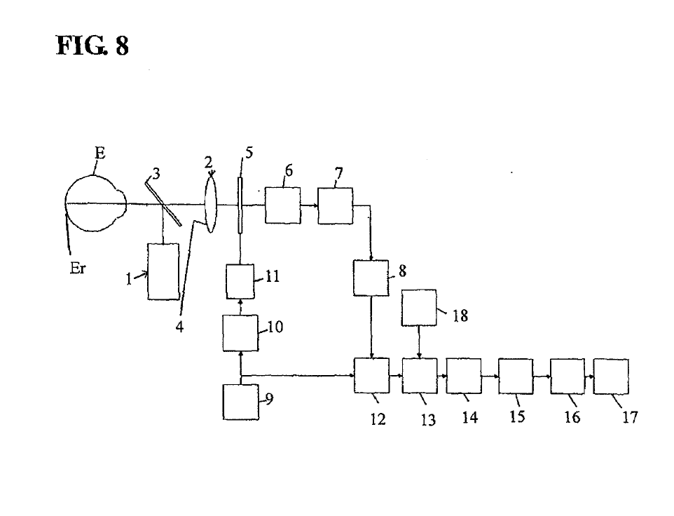

In the procedure described above, the method of distinguishing the pulse of the artery and vein of skewness, etc. is a method of sensing the lowest frames from a plurality of cardiac beats to get one cardiac beat or extracting one cardiac beat data in a time series from the lowest frames, and then executing skewness calculation or the like to make mapping. However, the detection of one cardiac beat can also utilize data obtained by sensing cardiac beats outside, such as electrocardiograms, as in the invention described in claim 8. The external synchronizing signals synchronizes with a pulse and takes a constant propagation delay time to carry strength and weakness of a beat to the arithmetic section. The arithmetic section senses a weak beat portion and extracts the second lowest frame from the lowest frame in consideration of the propagation delay time to be able to make a pulse data of one cardiac beat. The procedure in this method was shown in FIG. 8. In FIG. 8, reference numeral 18 is the detecting section for an external synchronizing signal.

### INDUSTRIAL APPLICABILITY

According to the present invention there is provided a blood flow rate imaging device that can display an arterial pulse part and a venous pulse part on the blood flow map. According to the present device, to say nothing of an artery and vein separation of a blood vessel, a low blood flow site and a site having a disorder are blackened on a blood flow map and an artery and vein also becomes black, whereby they are displayed black when superimposed and displayed, which makes easily understood a disorder site that likely generates a hematogenous disease. Accordingly, the blood flow rate imaging device of the present invention introduces a new measure into a method for evaluation of the blood flow of an eye ground, and is expected as an extremely beneficial clinically diagnostic tool.

1. A blood flow rate imaging device; comprising:
a laser beam irradiation system that irradiates a biological tissue having a blood cell with a laser beam;
a light receiving system having a light receiver including a large number of pixels that detects reflected light from the biological tissue;
an image capture section that continuously captures a plurality of images for a specified time that is one or more cardiac beats on the basis of a signal from the light receiver;
an image storage section that stores the plurality of images;
an arithmetic section that calculates a blood flow rate within the biological tissue from the time variation of the output signal of each pixel corresponding to the plurality of the stored images; and
a display section that displays the two-dimensional distribution of the calculation result as a blood flow map, wherein
the arithmetic section has a detecting section that detects an artery and a vein from a plurality of images of the one or more cardiac beats and distinguishably displays an arterial pulse part (artery map) and a venous pulse part (vein map) on the blood flow map of the display section.

2. The blood flow rate imaging device according to claim 1, wherein the detecting section calculates skewness (skew value) based on the variation of blood flow rates arranged in a time series for each pixel and detects an arterial pulse part and a venous pulse part.

3. The blood flow rate imaging device according to claim 1, wherein the above detecting section calculates the expected value of a probability density function by likening the variation of blood flow rates arranged in a time series for each pixel to the probability density function and detects an arterial pulse part and a venous pulse part.

4. The blood flow rate imaging device according to claim 1, wherein the detecting section calculates kurtosis based on the variation of blood flow rates arranged in a time series for each pixel and detects an arterial pulse part and a venous pulse part.

5. The blood flow rate imaging device according to claim 1, wherein the detecting section calculates a mode in which a probability density function is estimated to be a maximum by likening the variation of blood flow rates arranged in a time series for each pixel to a probability density function and detects an arterial pulse part and a venous pulse part.

6. The blood flow rate imaging device according to claim 2, wherein the detecting section statistically processes a peripheral blood flow value of one or more pixels for the blood flow value of each pixel including many statistical errors to calculate an average value and outputs one or more pulse components arranged in a time series with few noises needed for detecting the arterial pulse part and the venous pulse part.

7. The blood flow rate imaging device according to claim 2, wherein the detecting section averages time variations of the blood flow of each pixel over a plurality of cardiac beats for one cardiac beat and then extracts the pulse component.

8. The blood flow rate imaging device according to claim 2, wherein the detecting section cuts out one cardiac beat for the time variations of the blood flow of each pixel over a plurality of cardiac beats, based on a synchronization signal from an outside that synchronizes with a cardiac beat and then extracts a pulse component.

9. The blood flow rate imaging device according to claim 1, wherein, in the display section, an arterial pulse part is superimposed upon a venous pulse part on the blood flow map and displayed.

10. The blood flow rate imaging device according to claim 3, wherein the detecting section statistically processes a peripheral blood flow value of one or more pixels for the blood flow value of each pixel including many statistical errors to calculate an average value and outputs one or more pulse components arranged in a time series with few noises needed for detecting the arterial pulse part and the venous pulse part.

11. The blood flow rate imaging device according to claim 4, wherein the detecting section statistically processes a peripheral blood flow value of one or more pixels for the blood flow value of each pixel including many statistical errors to calculate an average value and outputs one or more pulse components arranged in a time series with few noises needed for detecting the arterial pulse part and the venous pulse part.

12. The blood flow rate imaging device according to claim 5, wherein the detecting section statistically processes a peripheral blood flow value of one or more pixels for the blood flow value of each pixel including many statistical errors to calculate an average value and outputs one or more pulse components arranged in a time series with few noises needed for detecting the arterial pulse part and the venous pulse part.

13. The blood flow rate imaging device according to claim 3, wherein the detecting section averages time variations of the blood flow of each pixel over a plurality of cardiac beats for one cardiac beat and then extracts the pulse component.

14. The blood flow rate imaging device according to claim 4, wherein the detecting section averages time variations of the blood flow of each pixel over a plurality of cardiac beats for one cardiac beat and then extracts the pulse component.

15. The blood flow rate imaging device according to claim 5, wherein the detecting section averages time variations of the blood flow of each pixel over a plurality of cardiac beats for one cardiac beat and then extracts the pulse component.

16. The blood flow rate imaging device according to claim 3, wherein the detecting section cuts out one cardiac beat for the time variations of the blood flow of each pixel over a plurality of cardiac beats, based on a synchronization signal from an outside that synchronizes with a cardiac beat and then extracts a pulse component.

17. The blood flow rate imaging device according to claim 4, wherein the detecting section cuts out one cardiac beat for the time variations of the blood flow of each pixel over a plurality of cardiac beats, based on a synchronization signal from an outside that synchronizes with a cardiac beat and then extracts a pulse component.

18. The blood flow rate imaging device according to claim 5, wherein the detecting section cuts out one cardiac beat for the time variations of the blood flow of each pixel over a plurality of cardiac beats, based on a synchronization signal from an outside that synchronizes with a cardiac beat and then extracts a pulse component.
